(19) United States
(12) Patent Application Publication
(10) Pub. No.: US 2026/0207132 A1
(43) Pub. Date: Jul. 23, 2026

(54) Oncology Sensor

(71) Applicant: BIOTRONIK SE & CO.KG, Berlin (DE)

(72) Inventors: Thomas DOERR, Berlin (DE); Volker LANG, Berlin (DE); Sebastian SCHULZE-LUCKOW, Berlin (DE); Frank BECKER, Berlin (DE)

(73) Assignee: BIOTRONIK SE & CO.KG, Berlin (DE)

(21) Appl. No.: 19/135,882

(22) PCT Filed: Nov. 15, 2023
(86) PCT No.: PCT/EP2023/081861

§ 371 Date: Jun. 5, 2025

(30) Foreign Application Priority Data
Dec. 19, 2022 (EP) 22214467.7

(51) Int. Cl. A61B 5/00; G16H 40/67

(52) U.S. Cl. CPC A61B 5/4836; A61B 5/746; G16H 40/67

(57) ABSTRACT

An apparatus for oncology therapy, the apparatus includes means for receiving physiological data of a patient undergoing oncology therapy according to at least one treatment parameter. The apparatus further includes means for providing at least one new and/or updated treatment parameter of the oncology therapy, based at least in part on the physiological data.

### CROSS-REFERENCE TO RELATED APPLICATIONS

This application is the United States National Phase under 35 U.S.C. § 371 of PCT International Patent Application No. PCT/EP2023/081861, filed on Nov. 15, 2023, which claims the benefit of European Patent Application No. 22214467.7, filed on Dec. 19, 2022, the disclosures of which are hereby incorporated by reference herein in their entireties.

### BACKGROUND

Acute and long-term side effects of multimodal oncology therapy significantly impair the quality of life and survival of patients with malignancies. Such multimodal therapies may include for example radiotherapy, classical (conventional) chemotherapy, immunotherapy and so-called targeted therapies.

Patients undergoing oncology therapy (e.g., chemotherapy, radiation, etc.) frequently develop cardiovascular complications during therapy and in the long-term course. Currently, patients are usually only attended cardiologically and/or cardiovascularly when the complications are already pronounced. However, the disadvantage of this strategy is a worsened prognosis compared with a strategy of detecting the complications as early as possible and mitigating them cardiologically and/or cardiovascularly and by adjusting oncology therapy.

Further, established tumor therapy strategies are joined by up to 30 new approvals per year with incompletely characterized side effect profiles. Many of these therapies have cardiotoxic side effects that may require complex treatment in the context of the patient's cardiac risk profile.

Therefore, there is still a need to further improve apparatuses, respective systems, computer programs and methods for oncology therapy.

The above need is at least in part met by the various aspects described herein.

The present disclosure is directed toward overcoming one or more of the above-mentioned problems, though not necessarily limited to embodiments that do.

### SUMMARY

According to one aspect of the invention, an apparatus for oncology therapy is provided. The apparatus comprises means for receiving physiological data of a patient undergoing oncology therapy according to at least one treatment parameter and means for providing at least one new and/or updated treatment parameter of the oncology therapy, based at least in part on the physiological data.

This may permit cost-effective, telemedical monitoring and therapy management for patients who are receiving or have received cardiotoxic oncological therapy, providing a suitable tool to adjust oncology treatment to the specific needs of the patient, increase the treatment success chances, and mitigate unwanted side effects. This may allow a continuous oncology therapy management to adjust the relevant treatment parameters when needed, which may be decisive in view of ever-changing therapies with a patient-specific severity of potentially, particularly cardiotoxic, side effects.

The apparatus may comprise means configured to receive physiological data of the patient, wherein the physiological data of the patient relate to the physiological condition of the patient such as at least one parameter associated with a health state of the patient. The apparatus may be a medical device, potentially (at least partly) implanted into the patient or worn by the patient, a (remote) server, a cloud-server, etc.

In an exemplary embodiment, the means for receiving physiological data may be adapted to receive the physiological data from an at least at least partly implantable and/or wearable sensor. This may be particularly advantageous as no patient interaction with the respective sensor is necessary and thus, continuous data acquisition for long-term assessment is facilitated.

In one example, when an electrocardiography, ECG, device may provide the physiological data, e.g., the apparatus may extract parameters from an ECG: Additionally or alternatively the ECG device may directly process the ECG waveform and provide extracted parameters to the apparatus. An ECG waveform typically comprises three characteristic components: a so-called P wave, which represents depolarization of the atria, a subsequent QRS complex, which represents depolarization of the ventricles, and finally a T wave, which represents repolarization of the ventricles. The extracted parameter(s) may for example comprise a QT-time, a PQ-time, a QRS-width, a variability of the heart rate, and/or data on whether, how often, and/or when bradycardia and breaks, atrial and/or ventricular extrasystoles (e.g., two subsequent ventricular extrasystoles (a couplet) or three subsequent ventricular extrasystoles (a burst)), high ventricular rates, breaks, cardiac inotropy, and/or tachycardia (e.g., atrial fibrillation/flutter with pre-excitation, atrial tachycardia, ventricular tachycardia, superventricular tachycardia, and/or torsades de pointes) occur.

In another example, the physiological data may alternatively or additionally be provided by a sensor measuring respiration-related parameters. These may for example comprise a respiratory frequency, depth, volume, and/or data on whether, how often, and/or when apnea and/or Cheyne-Stokes respiration occurs.

In another example, the physiological data may alternatively or additionally be provided by an accelerometer collecting motion-related parameters and might, e.g., indicate falling of the patient and/or other forms of motion.

It may further be expedient to consider psychological factors as many oncology patients suffer from the intense psychological burden associated with undergoing oncology therapy and especially the side effects that come along with it. Therefore, the apparatus may also receive measures for a psychological burden perceived by the patient which may be a result, e.g., of other physiological effects relating for example to cardiac inotropy, e.g., a weakening of the strength of the heart muscle contraction which may result in discomfort and psychological exertion. Such measures for the psychological burden perceived by the patient may for example be provided by patient input, e.g., via a remote device.

Generally, the physiological data may comprise non-static data that may vary on different timescales, e.g., in seconds to minutes, on a daily basis, or over years, such that the treatment parameters of the oncology treatment may be required to be adjusted in response to the variation of relevant non-static physiological data of the patient.

For example, oncology treatment administered to the patient may not only affect the cancer but bring along side effects. For example, the cardiovascular system of the patient may be affected by such side effects. Therefore, it may be advantageous to monitor associated physiological parameters, specifically cardiovascular parameters, as described herein to be aware of potential risks associated with these side effects and, if needed, adjust the treatment accordingly. In this sense, the apparatus may provide at least one new and/or updated treatment parameter of the oncology therapy, based at least in part on the physiological data.

Adjusting the therapy according to at least one new and/or updated treatment parameter of the oncology therapy may occur automatically. Generally, this adjustment may be made whenever a new and/or updated treatment parameter is provided. However, therapy may alternatively only be adjusted based on the new and/or updated treatment parameters, e.g., at a predetermined frequency and/or at predetermined times like for example once per hour, day, or week, or upon a patient's or physician's request. Additionally or alternatively, changing the therapy based on the updated and/or new parameter may occur, e.g., when a deviation of the updated treatment parameter from a previous treatment parameter exceeds a predetermined threshold and/or when a new treatment parameter is provided.

The apparatus for oncology therapy (and/or any part thereof) for evaluating how the at least one treatment parameter may be adapted based on the physiological data and/or parameter(s) may be designed as a server and/or cloud system.

In one exemplary embodiment, the physiological data may comprise at least one of an oncological staging parameter, at least one cardiovascular parameter, at least one respiratory parameter, at least one motion-related parameter, and at least one patient identification or personal patient data (e.g., age, gender, height, weight, disease).

These physiological data may advantageously provide a broad range of inputs based on which the treatment parameters may be adjusted for a well-adjusted therapy. This may increase oncology treatment success chances. Any of the data described herein may be provided by suitable sensors, e.g., implanted into the patient or worn by them for continuous data collection, by patient and/or health professional input, and/or by a data base with respective electronic patient files storing such data and making them accessible to the apparatus.

For example, the apparatus may comprise a (secure) interface to a patient data system, from which the at least one treatment parameter of the patient may be retrieved.

The oncological staging parameter of the patient may relate to the stage of the cancer, e.g., according to the following numerical stages: In stage 0 the cancer has not yet grown or spread from where it started. In stage 1 the cancer has not yet spread within the patient's body. It has grown but is still small. Stage 2 refers to a larger cancer that has grown but has not yet spread. A stage 3 cancer may have grown and spread into surrounding tissue, possibly to the lymph nodes. Finally, a stage 4 cancer may also be called metastatic as it has spread to at least one other organ. Additionally or alternatively, the staging may be according to any other staging system like, e.g., the TNM staging system, wherein T relates to the size of the cancer indicated by a number between 1 (small) and 4 (large), N relates to how many lymph nodes are showing metastases indicated by a number between 0 (none) and 3 (many), and M relates to whether the cancer has spread to other organs indicated by 0 (no) or 1 (yes). The at least one oncological staging parameter of the patient may, e.g., further relate to other diseases which might affect cancer development and/or treatment success chances. It may for example relate to the cancer health record, e.g., previously detected and/or treated cancers.

The at least one cardiovascular parameter of the patient may, e.g., relate to the health record of the patient and/or current parameters associated with the cardiovascular health status of the patient that, e.g., indicate a cardiovascular risk of the patient. Further, the at least one cardiovascular parameter may, e.g., comprise any cardiovascular parameter described herein, like, e.g., the QT-time, the PQ-time, the QRS-width, the variability of the heart rate, the patient's blood pressure, data on a cardiovascular medication of the patient, and/or data on whether, how often, and/or when bradycardia, atrial and/or ventricular extrasystoles, high ventricular rates, breaks, and/or tachycardia occur, and/or an ECG. It may for example further comprise any parameter associated with the cardiovascular system, e.g., data on stenoses etc.

The at least one respiratory parameter may comprise the respiratory frequency, depth, volume, and/or data on whether, how often, and/or when apnea and/or Cheyne-Stokes respiration occurs.

The at least one motion-related parameter may comprise any data recorded by an accelerometer as described herein and/or any parameter related to the motion of the patient in general, e.g., the amount of exercise and/or step count per day that might be recorded by a smart watch.

The personal patient data may for example comprise age, gender, weight, diseases. This data may also comprise further personal data, e.g., whether/how much the patient smokes or not, a history of ischemic disease, heart failure, cerebrovascular disease, diabetes, etc.

The physiological data may be linked to a patient, e.g. based on patient identifiers like, e.g., a name of the patient and/or a part thereof, a patient ID number, for example the patient's health insurance ID number, e.g. in an encrypted form.

In an exemplary embodiment, the at least one treatment parameter, the at least one new, and/or the at least one updated treatment parameter may comprise at least one of an oncological treatment scheme, a date, type, duration, and/or dose of a radiation, an oncological medication, and/or a cardiovascular medication. This may provide the advantage of adapting the treatment according to a broad range of treatment parameters for an ideal adjustment to the patient's needs.

The oncological treatment scheme may for example relate to chemotherapy, radiation therapy, stem cell therapy, biomarker testing for cancer treatment, hormone therapy, hyperthermia, immunotherapy, and/or any combination thereof. The treatment scheme may vary by adding and/or removing one of the treatments from the patient-specific plan according to a new and/or updated treatment parameter, and/or by adjusting the respective treatment according to the at least one new and/or updated treatment parameter. The scheme may then also provide the date, type, duration, and/or dose of the respective components employed in the chosen therapy.

Further, the date, type, duration, and/or dose are typical treatment parameters associated with the treatment schemes described herein and may be adjusted for radiation therapy or chemotherapy, for example.

In a further exemplary embodiment, the apparatus may further comprise an artificial intelligence-based assistant configured to provide the at least one new and/or updated treatment parameter of the oncology therapy, based at least in part on the physiological data.

The artificial intelligence-based assistant may provide an opportunity to, e.g., prevent other related issues: For example, a third of all patients with a left ventricular assist device (LVAD), suffer from right ventricular failures. This may be prevented by artificial intelligence-assisted therapy forms. Generally, this may not only improve the oncology therapy but also the operation of devices like LVADs for example.

The training data of the artificial intelligence-based assistant may comprise physiological data and treatment parameters that were successfully used in the past, for example, e.g. as stored in patient files, as used by the apparatus itself, and/or by other apparatus and may grow constantly for improved determination of the best new and/or updated treatment parameters.

Based on artificial intelligence and/or in a non-artificial intelligence-based apparatus, significant parameters may be determined for the respective patient in their situation such that the apparatus may only process that part of the physiological data that may be rated as significant and/or neglect insignificant parameters.

In an exemplary embodiment, the apparatus may be configured such that the at least one new and/or updated treatment parameter remains constant when at least one physiological parameter of the physiological data remains within a predetermined range. The predetermined range may be defined by a predetermined lower threshold and a predetermined upper threshold. The at least one new and/or updated treatment parameter may be adapted according to a first option when the at least one physiological parameter of the physiological data is below the predetermined range and the at least one new and/or updated treatment parameter may be adapted according to a second option when the at least one physiological parameter of the physiological data is above the predetermined range, respectively.

This may allow a robust adjustment scheme of the treatment parameters for an optimized treatment of the patient.

In one example, the first option may comprise reducing the at least one treatment parameter when the at least one physiological parameter of the physiological data is below the predetermined lower threshold and the second option may comprise increasing the at least one treatment parameter when the at least one physiological parameter of the physiological data is above the predetermined upper threshold. This may be beneficial when there is negative feedback from the at least one treatment parameter to the at least one physiological parameter such that the physiological parameter may be brought back into the predetermined range by reducing the according treatment parameter, e.g., the amount of medication administered to the patient may be reduced to increase the average heart rate when it was too low, or the average respiratory volume if it was too small. This may be repeated iteratively until the physiological parameter is within the interval between the predetermined lower and upper thresholds.

Vice versa, the first option may in an example comprise increasing the at least one treatment parameter when the at least one physiological parameter of the physiological data is below the predetermined lower threshold and the second option may comprise reducing the at least one treatment parameter when the at least one physiological parameter of the physiological data is above the predetermined upper threshold. This may be beneficial when there is positive feedback from the at least one treatment parameter to the at least one physiological parameter such that the physiological parameter may be brought back into the predetermined range by increasing the according treatment parameter, e.g., the amount of medication administered to the patient may be increased to increase the average heart rate when it was too low or the average respiratory volume if it was too small. This may be repeated iteratively until the physiological parameter is within the interval between the predetermined lower and upper thresholds.

The increase and/or decrease of the treatment parameter may directly correlate with a measure of how far the measured physiological parameter lies out of the ideal range defined by the predetermined upper and lower thresholds.

The first and second options may apply after one another, alternatingly, and/or as needed, e.g., in cases of overshooting when the correction of the at least one treatment parameter may have resulted from a change from a physiological parameter below the predetermined lower threshold to above the predetermined upper threshold or the other way round. Again, such subsequent adjustment according to the different options may be repeated iteratively until the at least one physiological parameter is within the interval between the predetermined lower and upper thresholds. This may be particularly advantageous when the predetermined lower and upper thresholds define narrow ranges.

The apparatus may accordingly be configured to find the maximum treatment effect at which selected side effects may remain below an acceptable value. In an exemplary titration, the at least one treatment parameter may be increased or decreased step-by-step until the at least one physiological parameter reaches a predetermined value (range) such as to approach an upper and/or lower limit. In an example, this may be implemented such that the amount of medication is increased until at least one of the physiological parameters associated with the health state of the patient indicates a decline of the patient's health with respect to that parameter, e.g., heart rhythm. Optionally, it may slightly be decreased subsequently, such as to reach an amount just below measurable side-effects occur.

A suitable example may for example be related to monitoring the left ventricular ejection fraction (LVEF). The LVEF may be monitored via echocardiography. A decrease of the LVEF by 10% or more may indicate an impairment of the cardiac function and require an adjustment of the oncology treatment as described herein.

In an exemplary embodiment, the apparatus may further be configured to provide an alarm signal, e.g., via a user interface, to the patient and/or a health professional and/or to automatically store the at least one treatment parameter, the at least one new, the at least one updated treatment parameter, and/or the physiological data of the patient in an electronic patient file.

Both improve the implemented feedback options in case of emergencies via the alarm signal and in terms of automatic documentation via data storing.

The alarm signal may for example be sent when at least one physiological parameter lies out of a predetermined safe range, the safe range may be wider than the ideal range between the predetermined lower and upper threshold, as described herein. The lower safe threshold may be below the predetermined lower threshold and the upper safe threshold may be above the predetermined upper threshold. The alarm signal may for example be a sound, a haptic signal, a text message, a warning light, etc. and may be communicated, e.g., via the remote device, the apparatus itself, any other device coupled to the apparatus, a user interface of any of those, etc.

The electronic data file may be updated continuously, e.g., once per minute, hour, day, week, etc. It may be a remote patient file stored in a patient data system, e.g., run by a network of health institutions. This may allow for continuous remote monitoring of the patient and/or to provide the data to the involved health professional(s) at any time when needed.

Depending on which parameter caused the apparatus to send such alarm signal, the alarm signal may be sent to the respective health professional, health institution, respective server, etc. For example, when the respective physiological parameter indicates a cardiac emergency, a cardiologist may be informed while an oncologist may be informed in case of a directly cancer-related emergency indicated by the at least one physiological parameter, e.g., via cancer-specific biomarkers. The apparatus may be configured to receive information on the respective responsible health professionals, e.g., with the patient information/identification.

In an example, the means for providing may be adapted to provide the at least one new and/or updated treatment parameter of the oncology therapy to a medical treatment device, preferably an at least partly implantable and/or wearable medical treatment device. Thus, the oncology therapy delivered by the medical treatment device may be adjusted in a fully automated manner, based on the at least one new and/or updated treatment parameter. Thus, an automized workflow limiting the amount of required intervention may be provided. The communication with the medical treatment device may be implemented as described herein.

Generally, the apparatus may be configured to transmit any data as encrypted data by means of a low-power transmission technology (medical implant communication service, MICS, Bluetooth (low energy), etc.) to a relay station (e.g., an apparatus-specific remote device, server, or application, a smartphone, etc.), which in turn is connected to the internet or a virtual private network for signaling exchange of all involved components, e.g., further medical devices, servers, etc. This may apply to any data transmission described herein. The encryption may for example increase the safety of such apparatuses and systems.

In an example, the apparatus may provide the at least one new and/or updated treatment parameter of the oncology therapy to a health professional, preferably in form of an instruction to adapt a setting of the medical treatment device manually.

Input from a health professional may yield an additional safety measure. The treatment parameters may be provided to one or more health professionals via a server.

Both options, providing the new and/or updated treatment parameter of the oncology therapy to a medical treatment device and to the health professional, may be combined, e.g., such that the at least one new and/or updated treatment parameter of the oncology therapy is automatically re-set, but the health professional is informed and/or asked to permit the re-set.

In an example, the apparatus may further be configured to repeatedly and/or periodically receive the physiological data of the patient undergoing oncology therapy according to the at least one treatment parameter. It may provide the at least one new and/or updated treatment parameter of the oncology therapy, based at least in part on the physiological data, such as to form a closed loop.

Such closed loop adaptation may provide well-adjusted treatment and reduce those times during which the treatment may be not ideal, e.g., in terms of side-effects. Thereby, it poses a suitable tool accounting for constantly changing conditions within the patient's body which require regular re-adjustment of the therapy for optimized treatment success chances.

The closed loop may for example comprise receiving the physiological data of the patient and providing the at least one new and/or updated treatment parameter, at predetermined times and/or at a predetermined frequency. Additionally or alternatively, the multiple closed loops may run consecutively at a high repetition rate until all treatment parameters are within the predetermined ranges defining optimized treatment conditions. When this is achieved, the repetition rate of the closed loops may be reduced until at least one physiological parameter increases or decreases such that it is not within the predetermined range anymore. Then, multiple closed loops may run consecutively at a high repetition rate again, as described herein, to readjust the treatment accordingly.

Additionally or alternatively, receiving the physiological data of the patient undergoing oncology therapy may be event-triggered, e.g., based on an event log of an electronic patient file.

In an exemplary embodiment, the apparatus may further be configured to adjust a measurement parameter, based at least in part on the physiological data and/or at least one of: the at least one treatment parameter, the at least one new and/or updated treatment parameter of the oncology therapy.

This may improve the over-all efficiency of the apparatus and the battery lifetime.

The measurement parameter may, e.g., comprise a frequency and/or a time interval determining when the physiological data may be acquired and/or received by the apparatus. For example, when the apparatus was not required to adjust the therapy according to at least one new and/or updated treatment parameter upon receiving the physiological data, the apparatus may, e.g., reduce the frequency at which the physiological data are received and evaluated for providing at least one new and/or updated treatment parameter of the oncology therapy. Vice versa, when substantial changes of the at least one new and/or updated treatment parameter of the oncology therapy were required, the frequency may be increased.

In an example, the apparatus may be a remote server and/or a cloud-based server.

In such exemplary embodiment, the functionalities of the apparatus may be controlled remotely, e.g., by a health professional, run fully automatic and/or without the patient noticing. This may increase patient comfort and satisfaction.

The physiological data, e.g., acquired by an implant of the patient may be sent to the remote server via a wireless connection and processed therein. The physiological data may further be (automatically) saved in a patient data base to be accessed by a health professional for continuous remote monitoring of the patient.

Additionally or alternatively, an apparatus for oncology therapy monitoring may be provided that comprises means for receiving physiological data of a patient undergoing oncology therapy according to at least one treatment parameter. It may further comprise means for evaluating the oncology therapy based at least in part on the physiological data. For example, it may be determined whether the physiological data is within one or more predetermined ranges, such as to assess whether any intended main effect of the therapy is achieved or whether side-effects are still within an acceptable range. The apparatus may send the evaluation to a device of the patient and/or doctor (computer, smartphone, etc.), such as to provide a monitoring of the therapy.

According to another aspect of the invention, a system may comprise an apparatus as described herein and optionally a medical sensor for providing the physiological data to the apparatus, and/or a medical treatment device for treatment based on the at least one new and/or updated treatment parameter of the oncology therapy is provided.

This may yield the advantageous combination of such apparatus, medical sensor, and/or medical treatment device, which, in combination, may provide a well-adjusted treatment to the patient, based at least in part on the physiological parameters describing the current health state of the patient.

The medical sensor may be any medical device suited to record at least one physiological parameter associated with the patient. The medical sensor may for example be a cardiac sensor, an accelerometer, a neurostimulation device, an ECG, etc. The sensor may, for example, be a wearable and/or an at least party implantable device (e.g., a cardiac implant, such as a pacemaker, cardioverter-defibrillator, etc.).

The medical treatment device may be any device configured to provide a treatment to the patient. The treatment may for example be an oncology treatment as described herein and/or another treatment, for example of the cardiovascular or the respiratory system of the patient, e.g., a pacemaker, an implantable cardiac resynchronization therapy defibrillator, an LVAD, a (non-intravenous) implantable cardioverter-defibrillator, an implantable pulse generator, a ventricular assist device, an extracorporeal membrane oxygenation device, a neurostimulation device, etc. The apparatus may be configured to provide the new and/or updated treatment parameter to the medical treatment device.

In general, any of the exemplary devices and/or apparatus may be comprised by the system, and the device and/or apparatus may comprise the functionalities as described herein. For example, the system may comprise a device of the patient and/or doctor, as outlined herein.

The same device may in some examples be the medical sensor and the medical treatment device. The communication between any of the constituents of the system may for example be directed via a remote device functioning as a relay station.

The functions described herein may for example be implemented within one apparatus which, e.g., functions as a drug pump delivering a treatment in form of a drug, records physiological data, and adjusts the way in which the drug is released into the patient's body based on the as-acquired physiological data. Thereby, the whole system may be implemented in one apparatus.

In another example, the system may comprise at least one medical sensor comprising at least one of: an ECG, a neurostimulation device, and an accelerometer. Additionally or alternatively, the at least one medical sensor may be an at least partly implantable device and/or a wearable device.

The invention may be particularly advantageous for implants and wearable device as they inherently offer the possibility to continuously monitor at least one physiological parameter of the patient and/or to continuously deliver a treatment according to the at least one treatment parameter. The devices or parts thereof may alternatively be attached to the patient's skin as a piercing and/or glued or attached in any other way to the patient's skin.

An ECG device, a neurostimulation device, and an/or accelerometer may be implanted into the patient or be worn by the patient as wearable devices in addition or alternatively to each other. Each of the devices may provide at least one physiological parameter associated with the patient, based on which the apparatus may provide the at least one new and/or updated treatment parameter to the medical treatment device. As described herein, this entire process may run completely automatically and/or with input from a health professional.

An exemplary system for cardiac tumor treatment, e.g., comprising delivery of oncological drugs for cardiac tumor treatment via the bloodstream, may comprise at least one cardiac/cardiovascular sensor and a medical drug pump/drug delivery device. The heart sensor may for example be configured to communicate with the drug delivery device and the system may determine the oncological drug delivery via the drug delivery device. This may account for the main symptoms of cardiac tumors, comprising an increase in blood pressure, formation of blood clots, cardiac arrhythmias, heart attacks and heart failure.

Any function described in reference to any of the constituents of such system may be implemented in full or in part in any other constituent of the system described herein, for example in a remote device which may be, e.g., a mobile phone running a respective application.

According to an aspect of the invention, a computer program is provided. The computer program may comprise instructions which, when the program is executed, causes an apparatus for oncology therapy to receive physiological data of a patient undergoing oncology therapy according to at least one treatment parameter and provide at least one new and/or updated treatment parameter of the oncology therapy, based at least in part on the physiological data.

Implementing these functions instructions of a computer programme yields all the advantages described in reference to the respective apparatuses and systems comprising further devices. The computer program may further comprise instructions which, when the programme is executed, cause the respective apparatus or any other constituent of the respective system to execute functions described in reference to such said apparatus and/or system.

In an exemplary embodiment, the computer program may comprise further instructions which, when the program is executed, cause an apparatus for oncology therapy to repeatedly and/or periodically receive the physiological data of the patient undergoing oncology therapy according to the at least one treatment parameter and to provide the at least one new and/or updated treatment parameter of the oncology therapy, based at least in part on the physiological data, such as to form a closed loop.

Implementing such closed loop in instructions of a computer programme advantageously increases the degree of automatization and thus reduces the required input by any users, e.g., health professionals.

According to a further aspect of the invention, a method is provided. The method comprises receiving physiological data of a patient undergoing oncology therapy according to at least one treatment parameter, and providing at least one new and/or updated treatment parameter of the oncology therapy, based at least in part on the physiological data. The method may be carried out by the apparatus as described herein, for example.

Such method poses an advantageous approach to adjust an oncology treatment to state of the patient, measured in terms of physiological parameters.

In an example, the method further comprises repeatedly and/or periodically receiving the physiological data of the patient undergoing oncology therapy according to the at least one treatment parameter and providing the at least one new and/or updated treatment parameter of the oncology therapy, based at least in part on the physiological data, such as to form a closed loop.

This closed loop method yields all advantages described herein in reference to respective closed loop apparatus is, systems, and computer programs.

Additionally or alternatively a method may be provided for oncology monitoring, comprising receiving physiological data of a patient undergoing oncology therapy according to at least one treatment parameter. It may further comprise evaluating the oncology therapy based at least in part on the physiological data. The method may be carried out by the apparatus as described herein, for example.

For example, it may be determined whether the physiological data is within one or more predetermined ranges, such as to assess whether any intended main effect of the therapy is achieved or whether side-effects are still within an acceptable range. The method may further comprise sending the evaluation to a device of the patient and/or doctor (computer, smartphone, etc.), such as to provide a monitoring of the therapy.

In an example, the method further comprises repeatedly and/or periodically receiving the physiological data of the patient undergoing oncology therapy according to the at least one treatment parameter and evaluating the physiological data.

It is noted that all aspects outlined herein may be implemented as method steps and/or instructions of a computer program and/or corresponding functions of an apparatus, even if described only with respect to a method, a computer program or an apparatus.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 A schematic representation of a system comprising a medical apparatus communicating with a remote device, a medical pump, a patient data system, and servers at medical institutions via a cloud-based server.

FIG. 2 A schematic representation of a collection of apparatuses incorporated into the same system and implanted into the patient.

### DETAILED DESCRIPTION

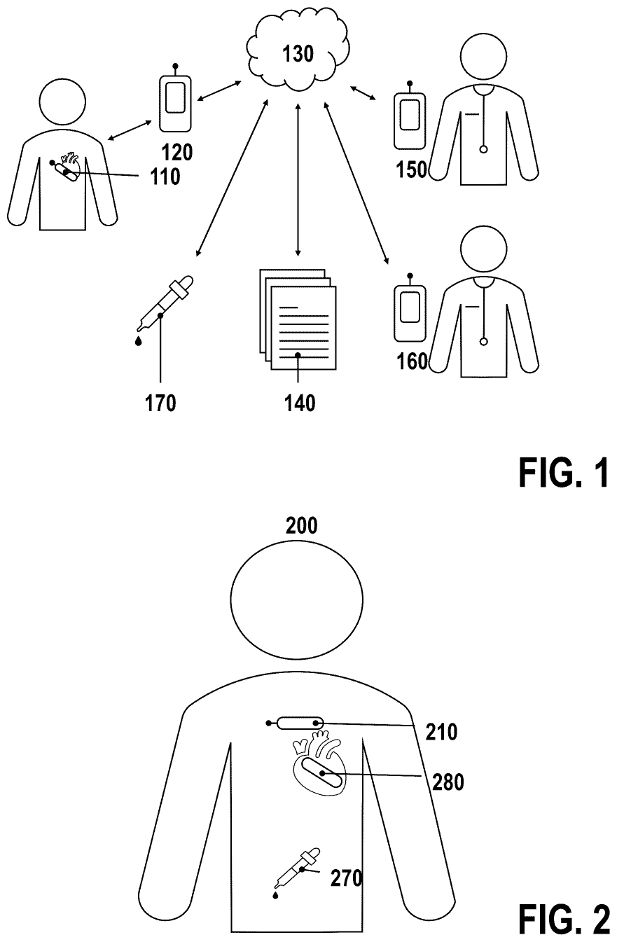

FIG. 1 shows a schematic representation of a system 100 comprising an apparatus 110, a remote device 120, a remote server 130, a patient data system 140 (e.g. an electronic patient file), a first remote (medical) server 150, a second remote (medical) server 160, and a medical treatment 170.

The exemplary apparatus 110 is an implant of any kind implanted into the patient undergoing oncology therapy 170. In this example, the apparatus 110 (e.g. a medical sensor) is located at the heart of the patient and may collect medical device data relating to the cardiac system of the patient. In FIG. 1, the subcutaneously implanted monitoring implant 110 records a 30-second ECG recording several times a day (e.g., every 6 hours) and, in parallel, a 30-second respiratory waveform (impedance-based) and transmits them to a patient relay 120 via a telemetry interface. In addition, the apparatus or medical sensor 110 (e.g. a sensor implant) may be equipped with detection algorithms that can detect tachycardic and bradycardic arrhythmias and then transmit event-triggered recordings as well. Further, statistics on extrasystoles, motion trajectories, atrial burdens, body temperature, etc. are recorded and transmitted, e.g., once a day.

The exemplary apparatus 110 of FIG. 1 is configured to exchange signaling with a remote device 120 which may for example be the mobile phone or a smart watch of the patient running a medical device-associated application. The apparatus 110 and the remote device 120 of FIG. 1 comprise means for wireless communication. In other examples, they may be connected via a wire and/or any other means for signal transmission or all functions of the remote device 120 may be transferred to other parts of the system 100 such that no remote device 120 is needed and the apparatus 110 may communicate directly with the other parts of the system 100. In the shown example, signaling is transmitted from the apparatus 110 via the remote device 120 to a remote server 130 and vice versa, which is cloud-based in the schematically shown example. The patient relay device 120 can be designed as an apparatus-specific remote device, server, or application, a smartphone app. It then forwards the data via the telemetry interface to the cloud-based remote server 130.

In the exemplary embodiment of FIG. 1, the remote server 130 provides a data exchange platform connecting the medical device 110 (via the remote device 120) with all other constituents 140, 150, 160, 170 of the system 100. Additionally or alternatively, any other connection system with direct connections/signaling exchange between at least some of constituents of the system 100 is possible and/or the remote server 130 may be implemented in form of a software running on any of the constituents of the system 100 shown in the exemplary embodiment of FIG. 1.

Further, in the exemplary system 100 of FIG. 1, signaling is exchanged via the remote server 130 with two external medical servers 150, 160 which may be servers at a hospital, doctor's office and/or any other health institution, e.g. related to a cardiologist and an oncologist, each responsible for a part of the treatment of the patient. In other examples, these servers may be any other servers not associated with any health institution as well. There may be more or less of such servers 150, 160 in the system 100.

The involved health professional(s) may receive new and/or updated treatment parameter(s) of the oncology therapy 170, based at least in part on the physiological data acquired by the apparatus 110 via the servers 150, 160. They may then for example adjust the treatment 170 accordingly by re-setting at least one parameter of the medication, operation, and/or radiation (e.g., date, type, duration, and/or dose). This may also occur automatically via the remote server 130 without input from a health professional, the patient, and/or any other user of the system 100.

The physiological data recorded by the apparatus 110, the treatment parameter(s) calculated and/or suggested based thereon, and/or the actual re-set treatment parameters according to input from health professionals may be stored (automatically) in the patient data system 140 comprising at least one electronic file per patient storing the data described herein, possibly additionally to further patient-related data, e.g., patient information like gender, age, height, weight, etc.

Any exchange of signaling in the system 100 as described herein may occur automatically and/or in response to user input. The signaling may for example be transmitted wirelessly (e.g., via Bluetooth or Wi-Fi) or via wires or other electrical conductors.

The apparatus 110 may perform all functions of receiving physiological data of a patient undergoing oncology therapy 170 according to at least one treatment parameter and providing at least one new and/or updated treatment parameter of the oncology therapy, based at least in part on the physiological data to the remote server 130. Alternatively, in the schematically shown system of FIG. 1, the server running the remote server 130 may be seen as the apparatus described herein, e.g. the remote server 130 may evaluate the provided physiological data and provide the at least one new and/or updated treatment parameter. Any combination thereof is also possible such that these functions are distributed to both, the apparatus 110 and the remote server 130. In an even further alternative, these functions may alternatively or additionally be at least in part performed by further constituents of the system 100.

FIG. 2 exemplarily shows a collection of apparatuses 210, 270, 280 incorporated into the same system 200 and implanted into the patient. In detail, the system 200 comprises a medical sensor 210. It may further comprise a medical pump 270 for a treatment, and/or another medical treatment device 280, e.g. a ventricular assist device. The cooperation of these devices 210, 270, 280 may optimize the efficiency of the whole system as described in the following:

The apparatus 210 may, as described herein, be configured to collect physiological data concerning the current health state of the patient to provide frequently updated physiological data based on which the treatment parameters may be updated. The medical pump 270 for a treatment, and/or the ventricular assist device 280 may be re-set according to the treatment parameters in a closed-loop configuration or based on user input as described herein to continuously provide a treatment adjusted to the patient's needs.

The medical pump 270 for a treatment, and the ventricular assist device 280 are only examples for apparatuses included in an according system 200 and other and/or additional devices may be included as well. The communication with other components may for example be realized according to the exemplary embodiment of FIG. 1.

The system 200, e.g. apparatus 210, is designed in such a way that it performs the evaluation of the sensor data depending on the oncological therapy parameters (dose, time, history, etc.) and the cardiovascular risk factors. Thus, risk parameters stemming from specific drugs are weighted higher than cardiac findings not related to the therapy in order to establish a higher specificity of causality of the oncological therapy with the cardiac side effects. For example, anthracyclines, histone deacetylase inhibitors, tyrosine kinase inhibitors, and arsenic trioxide are associated with the expression of torsade de pointes arrhythmias. When these groups of drugs are used, the observation of the measured Q-T time is weighted highly as an early detection and, depending on the specific drug, may lead to an alert to the first and second treatment provider, e.g., the respective health professional, e.g. in case it is outside a predetermined range. For example, one or more updated and/or new treatment parameters may be provided with the alert, so that the medication dose can be adjusted if necessary or an alternative cardiovascular and/or oncological treatment regimen is selected. In some example, the alert may indicate that the therapy needs to be stopped.

Additionally or alternatively, the evaluation system can also control a medication pump 270 in such a way that, in the event of, e.g., an acute cardiotoxic reaction, an interruption of the medication administration or a dose adjustment is automatically carried out.

In some examples, the system 200 may be provided such that it comprises an implantable cardiac sensor device. At least in part based on the physiological data provided by the cardiac sensor device, at least one new and/or updated treatment parameter for oncological treatment may be provided.

The system 200 may additionally or alternatively comprise an implantable medical pump for providing an oncological treatment. For example, the (oncological) treatment by the pump may be adjusted accordingly based at least in part based on the new and/or updated treatment parameter, e.g. as outlined herein.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An apparatus for oncology therapy, the apparatus comprising:
means for receiving physiological data associated with a health state of a patient undergoing oncology therapy according to at least one treatment parameter; and
means for providing at least one new and/or updated treatment parameter of the oncology therapy, based at least in part on the physiological data.

2. The apparatus of claim 1, wherein the physiological data comprise at least one of:
a. an oncological staging parameter,
b. at least one cardiovascular parameter,
c. at least one respiratory parameter,
d. at least one motion-related parameter.

3. The apparatus of claim 1, wherein the at least one treatment parameter, the at least one new, and/or the at least one updated treatment parameter comprises at least one of:
a. an oncological treatment scheme,
b. date, type, duration, and/or dose of a radiation,
c. date, type, duration, and/or dose of an oncological medication,
d. date, type, duration, and/or dose of a cardiovascular medication.

4. The apparatus of any of the preceding claims, further comprising an artificial intelligence-based assistant configured to provide the at least one new and/or updated treatment parameter of the oncology therapy.

5. The apparatus of claim 1, the at least one new and/or updated treatment parameter remains constant when at least one physiological parameter of the physiological data remains within a predetermined range;
wherein the at least one new and/or updated treatment parameter is adapted according to a first option when the at least one physiological parameter of the physiological data is below the predetermined range; and
wherein the at least one new and/or updated treatment parameter is adapted according to a second option when the at least one physiological parameter of the physiological data is above the predetermined range.

6. The apparatus of claim 1, further configured to provide an alarm signal to the patient and/or a health professional; and/or automatically store the at least one treatment parameter, the at least one new, the at least one updated treatment parameter, and/or the physiological data of the patient in a patient data system.

7. The apparatus of claim 1, wherein the means for providing are adapted to provide the at least one new and/or updated treatment parameter of the oncology therapy to a medical treatment device, preferably an at least partly implantable and/or wearable medical treatment device.

8. The apparatus of claim 1, wherein the means for receiving physiological data are adapted to receive the physiological data from an at least partly implantable and/or wearable sensor.

9. The apparatus of claim 1, further configured to repeatedly and/or periodically:
receive the physiological data of the patient undergoing oncology therapy according to the at least one treatment parameter, and
provide the at least one new and/or updated treatment parameter of the oncology therapy, based at least in part on the physiological data, such as to form a closed loop.

10. The apparatus of claim 1, further comprising means for adjusting a measurement parameter for the physiological data, based at least in part on received physiological data, the at least one treatment parameter, the at least one new treatment parameter, and/or the at least one updated treatment parameter.

11. The apparatus of claim 1, wherein the apparatus comprises a server.

12. A system comprising:
an apparatus according to claim 1 and at least one of:
a medical sensor for providing the physiological data to the apparatus;
a medical treatment device for treatment based on the at least one new and/or updated treatment parameter of the oncology therapy.

13. The system of claim 12, wherein:
the at least one medical sensor comprises at least one of: an electrocardiography, ECG, device; a neurostimulation device; and an accelerometer; and/or
the at least one medical sensor is an at least partly implantable device and/or a wearable device.

14. A computer program comprising instructions which, when the program is executed, cause an apparatus for oncology therapy to receive physiological data associated with a health state of a patient undergoing oncology therapy according to at least one treatment parameter; and provide at least one new and/or updated treatment parameter of the oncology therapy, based at least in part on the physiological data.

15. The computer program according to claim 14, comprising further instructions which, when the program is executed, cause the apparatus for oncology therapy to repeatedly and/or periodically receive the physiological data of the patient undergoing oncology therapy according to the at least one treatment parameter and to provide the at least one new and/or updated treatment parameter of the oncology therapy, based at least in part on the physiological data, such as to form a closed loop.
